(19) United States
(12) Patent Application Publication
(10) Pub. No.: US 2022/0008145 A1
(43) Pub. Date: Jan. 13, 2022

(54) VIRTUAL POINTER FOR REAL-TIME ENDOSCOPIC VIDEO USING GESTURE AND VOICE COMMANDS

(71) Applicant: Jack Wade, LA JOLLA, CA (US)

(72) Inventor: Jack Wade, LA JOLLA, CA (US)

(21) Appl. No.: 17/325,015

(22) Filed: May 19, 2021

Related U.S. Application Data
(60) Provisional application No. 63/029,324, filed on May 22, 2020.

(51) Int. Cl. A61B 34/00; A61B 34/20; A61B 17/00; G16H 40/20; G16H 10/60; G16H 40/67; G06T 7/10; G06T 7/00

(52) U.S. Cl. CPC A61B 34/25; G06T 2207/30004; A61B 17/00234; G16H 40/20; G16H 10/60; G16H 40/67; G06T 7/10; G06T 7/0012; A61B 2560/0493; A61B 2034/2068; A61B 2034/2074; A61B 2017/00207; A61B 2017/00973; A61B 2017/00115; G06T 2207/10068; G06T 2207/20084; A61B 34/20

(57) ABSTRACT

The present invention pertains to the need to precisely identify and communicate a specific position on tissue or an organ with a Virtual Pointing Device (VPD) software tool. The VPD uses a combination of audio key words and the surgeon's hand movements to invoke various functionality.

### PRIORITY CLAIMS

This application claims the benefit of U.S. Provisional Application Ser. No. 63/029,324, filed on May 22, 2020, the contents of which are incorporated herein.

### BACKGROUND OF THE INVENTION

A majority of surgical procedures are performed using a technique called minimally invasive surgery (MIS) wherein a video camera and the surgical instruments are inserted into the body cavity through small openings called portals. MIS is less traumatic on the patient and results in quicker recovery times.

Advances in video technology, computer enhanced vision, and artificial intelligence enable better and faster procedures. However, MIS techniques such as endoscopic and laparoscopic surgeries, where there is only an indirect view of the operative field, present new challenges. For example, unlike open surgeries, there is no easy way for the surgeon to point by gesture to an observed tissue anomaly or physical artifact. The ability to direct and focus viewer attention by gesture is one of the most intuitive and important means for a surgeon to convey accurate information to support staff, consulting surgeons and specialists who are witnessing a live procedure over video. Hand gestures are also a convenient means of interacting with supporting equipment.

New video assistive tools and methods continue to evolve and are enabled by ever more sophisticated graphical user interfaces and software using mathematical algorithms, artificial intelligence, and augmented reality. Previously described, the Software Tools Platform for Medical Environments (U.S. Pat. No. 9,526,586B2), a.k.a. Surgeons Video Tool Kit (SVTK) is an example that provides a continuously expanding set of software tools. Many of these tools were pioneered in sophisticated graphical user interface software and video games. The tools provide enhanced vision, instant analysis, and extend human perception and analysis beyond human capability.

Many of these innovations depend upon the hand-eye coordination capabilities of the surgeon. Most surgical procedures require a surgeon to use and coordinate two instruments simultaneously, controlled by the left and right hands. Because both hands are busy, user interfaces such as voice commands, foot pedals, body gestures, even computer monitored eye movement are used to direct assistance from computers, electronic monitoring devices, and supporting staff.

### SUMMARY OF THE INVENTION

The present invention seeks to address the need to precisely identify and communicate a specific position on tissue or an organ with a Virtual Pointing Device (VPD) software tool. The VPD uses a combination of audio key words and the surgeon's hand movements to invoke various functionality. For example, the VPD can be used to overlay a synthetic visual dotted line starting from the end of the selected instrument and extending a specified distance in the direction the instrument is pointed. The VPD accomplishes this by analyzing the live endoscopic video and calculating the direction that the surgical instrument is pointing. Depending on the instrument employed, the system determines whether to use edge detection algorithms for resolving direction, or artificial intelligence, or a model derived from physical specifications and/or artificial intelligence neural networks trained with visual observations of the instrument.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

FIG. 1 is an image of the VPD software tool of present invention.

### DETAILED DESCRIPTION OF THE PREFERRED EMBODIMENT

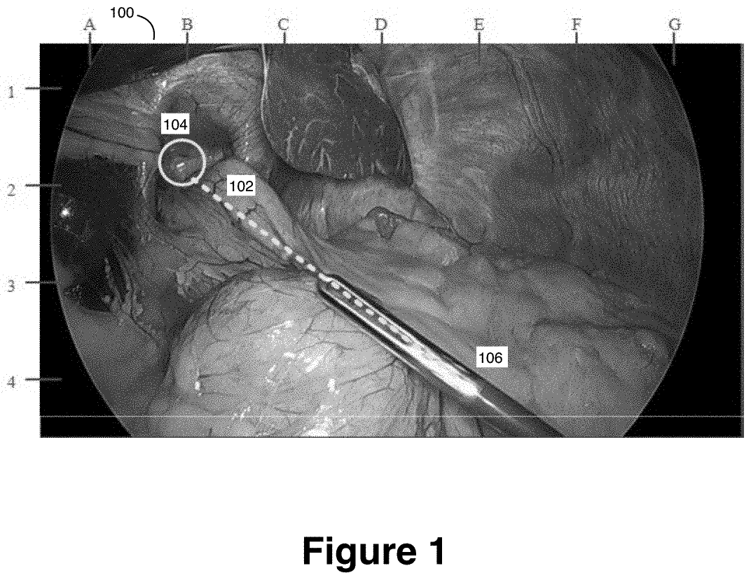

FIG. 1 is a video image 100 of the VPD software tool of present invention illustrating how the VPD software tool extends a yellow synthetic line from the direction the surgical probe is pointing. The preferences for line formats 102, end caps 104, and animation 106 for each instrument are stored in a database. The surgeon controls the animated action of a VPD by issuing commands via voice or other means. The example in FIG. 1 might be created with commands: “POINTER ON, LINE (LONGER, SHORTER, BRIGHTER, DIMMER, COLOR RED), CIRCLE (BIGGER, SMALLER)” and so on.

The VPD uses a combination of audio key words and the surgeon's hand movements to invoke various functionality. For example, the VPD can be used to overlay a synthetic visual dotted line starting from the end of the selected instrument and extending a specified distance in the direction the instrument is pointed. The VPD accomplishes this by analyzing the live endoscopic video and calculating the direction that the surgical instrument is pointing. Depending on the instrument employed, the system determines whether to use edge detection algorithms for resolving direction, or artificial intelligence, or a model derived from physical specifications and/or artificial intelligence neural networks trained with visual observations of the instrument.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

Embodiments presented are particular ways to realize the invention and are not inclusive of all ways possible. Therefore, there may exist embodiments that do not deviate from the spirit and scope of this disclosure as set forth by appended claims, but do not appear here as specific examples. It will be appreciated that a great plurality of alternative versions are possible.

What is claimed is:

1. A medical software tools system, comprising:
a tool for sensing surgeon hand movements in connection with surgical camera usage to invoke a video overlay displaying a synthetic visual path starting from an end of a selected instrument onward through an intended path of movement in a specified distance and direction corresponding to an orientation of said tool;
a computer system receiving an image stream from said surgical camera;
said computer system providing a user interface overlay adapted for presentation over an surgical image stream and analyzing said surgical image steam and calculating an anticipated direction of movement corresponding to said direction said surgical tool is oriented; and
use of a combination of audio keywords and movements to enable predictive surgical tool movements to assist said surgeon.

2. A system according to claim 1 wherein a foot pedal is utilized to enable surgeon visual displays.

3. A system according to claim 1 wherein a virtual pointing device is enabled by said computer by accessing patient data corresponding with organ placement within said patient.

4. A system according to claim 3 wherein said virtual pointing device is enabled to overlay a synthetic visual dotted line starting from said end of the selected surgical tool and extending a specified distance in a direction said selected surgical tool the is pointed.

5. A system according to claim 1 wherein said computer determines whether to use edge detection algorithms for resolving direction, or an artificial intelligence function derived from physical specifications including artificial intelligence neural networks trained with visual observations of said surgical tool.

6. A system according to claim 1 wherein a cloud network collects surgical images on an enterprise scale to enable hospitals to ingest, manage, and fully utilize patient surgical video within a hospital network and to share said video with designated users and to automatically record video during surgery and provide key clips for evaluation of hospital resources and to store said video for integration with patient electronic health records.

7. A system according to claim 6 for hospital use for risk mitigation.

8. A system according to claim 6 for hospital use for improving hospital quality standards.

9. A system according to claim 1 wherein detection of tumor margins identified by comparing color spectral changes and rates of color spectral change in real time in a diseased area with said color spectral changes and said rates of color spectral change occurring in adjacent or surrounding proximate areas where said areas are identified by finding sets of readings with similar spectral signatures or rates of change.

10. A method of using medical software tools system, comprising:
a tool for sensing surgeon hand movements in connection with surgical camera usage to invoke a video overlay displaying a synthetic visual path starting from an end of a selected instrument onward through an intended path of movement in a specified distance and direction corresponding to an orientation of said tool;
a computer system receiving an image stream from said surgical camera;
said computer system providing a user interface overlay adapted for presentation over a surgical image stream and analyzing said surgical image steam and calculating an anticipated direction of movement corresponding to said direction said surgical tool is oriented; and
use of a combination of audio keywords and movements to enable predictive surgical tool movements to assist said surgeon.

11. A method according to claim 1 wherein a foot pedal is utilized to enable surgeon visual displays.

12. A method according to claim 1 wherein a virtual pointing device is enabled by said computer by accessing patient data corresponding with organ placement within said patient.

13. A method according to claim 3 wherein said virtual pointing device is enabled to overlay a synthetic visual dotted line starting from said end of the selected surgical tool and extending a specified distance in a direction said selected surgical tool the is pointed.

14. A method according to claim 1 wherein said computer determines whether to use edge detection algorithms for resolving direction, or an artificial intelligence function derived from physical specifications including artificial intelligence neural networks trained with visual observations of said surgical tool.

15. A method according to claim 1 wherein a cloud network collects surgical images on an enterprise scale to enable hospitals to ingest, manage, and fully utilize patient surgical video within a hospital network and to share said video with designated users and to automatically record video during surgery and provide key clips for evaluation of hospital resources and to store said video for integration with patient electronic health records.

16. A method according to claim 6 for hospital use for risk mitigation.

17. A method according to claim 6 for hospital use for improving hospital quality standards.

18. A method according to claim 1 wherein detection of tumor margins identified by comparing color spectral changes and rates of color spectral change in real time in a diseased area with said color spectral changes and said rates of color spectral change occurring in adjacent or surrounding proximate areas where said areas are identified by finding sets of readings with similar spectral signatures or rates of change.

19. A medical software tools system, comprising:
a tool for sensing surgeon hand movements in connection with surgical camera usage to invoke a video overlay displaying a synthetic visual path starting from an end of a selected instrument onward through an intended path of movement in a specified distance and direction corresponding to an orientation of said tool;
a computer system receiving an image stream from said surgical camera; said computer system providing a user interface overlay adapted for presentation over an surgical image stream and analyzing said surgical image steam and calculating an anticipated direction of movement corresponding to said direction said surgical tool is oriented;
use of a combination of audio keywords and movements to enable predictive surgical tool movements to assist said surgeon; and
a cloud network collects surgical images on an enterprise scale to enable hospitals to ingest, manage, and fully utilize patient surgical video within a hospital network and to share said video with designated users and to automatically record video during surgery and provide key clips for evaluation of hospital resources and to store said video for integration with patient electronic health records.

20. A system according to claim 19 wherein detection of tumor margins identified by comparing color spectral changes and rates of color spectral change in real time in a diseased area with said color spectral changes and said rates of color spectral change occurring in adjacent or surrounding proximate areas where said areas are identified by finding sets of readings with similar spectral signatures or rates of change.
